(19) United States
(12) Patent Application Publication
(10) Pub. No.: US 2015/0132256 A1
(43) Pub. Date: May 14, 2015

(54) DETECTING AND MONITORING MUTATIONS IN HISTIOCYTOSIS

(71) Applicant: Trovagene, Inc., San Diego, CA (US)

(72) Inventors: Filip Janku, Houston, TX (US); Mark G. Erlander, Carlsbad, CA (US); Cecile Rose Vibat, San Diego, CA (US); Karena Kosco, Carlsbad, CA (US); Abdel-Wahab Omar, New York, NY (US); Eli L. Diamond, New York, NY (US); David M. Hyman, New York, NY (US)

(21) Appl. No.: 14/517,847

(22) Filed: Oct. 18, 2014

Related U.S. Application Data
(60) Provisional application No. 61/893,218, filed on Oct. 19, 2013.
(60) Provisional application No. 61/977,611, filed on Apr. 9, 2014.
(60) Provisional application No. 62/006,260, filed on Jun. 1, 2014.
(60) Provisional application No. 62/040,366, filed on Aug. 21, 2014.

(51) Int. Cl. C12Q 1/68

(52) U.S. Cl. CPC C12Q 1/6883; C12Q 2600/156; C12Q 2600/118
USPC 424/85.2; 514/300; 435/6.12; 435/6.11; 506/2

(57) ABSTRACT

Provided is methods of detecting a mutation in a histiocytosis patient. Also provided is methods of selecting and/or applying treatment or therapy for a histiocytosis patient. Further provided is a method of treating a patient having a histiocytosis.

### CROSS-REFERENCE TO RELATED APPLICATIONS

This application claims the benefit of U.S. Provisional Application No. 61/893,218, filed Oct. 19, 2013, U.S. Provisional Application No. 61/977,611, filed Apr. 9, 2014, U.S. Provisional Application No. 62/006,260, filed Jun. 1, 2014, and U.S. Provisional Application No. 62/040,366, filed Aug. 21, 2014, all incorporated by reference in their entirety.

### BACKGROUND OF THE INVENTION

(1) Field of the Invention

This present invention is directed to methods of determining treatment options, and methods of treating, histiocytosis. More specifically, the invention provides improved assays for detecting and quantitating mutations associated with histiocytosis, and the determination of treatment options that utilize the results of those assays.

(2) Description of the Related Art

Histiocytosis is a group of rare diseases characterized by the proliferation of histiocytes, or cells derived from monocytes, e.g., tissue macrophages and dendritic cells. Three groups of histiocytosis are recognized. One group is macrophage disorders, including hemophagocytic lymphohistiocytosis and Rosai-Dorfman Disease. The second group is malignant histiocytosis, and the third group is dendritic cell disorders, including Langerhans cell histiocytosis (LCH), juvenile xanthogranuloma, and Erdheim-Chester disease (ECD). ECD is a rare form of non-Langerhans cell histiocytosis affecting adults, which is associated with xanthogranulomatous infiltration of foamy macrophages (Janku et al., 2010, 2013; Arnaud et al., 2011)

The V600E mutation in BRAF and other mutations in the RAS-RAF-MEK-ERK and RAS-PI3K-AKT signaling pathways, including mutations in KRAS, PIK3A, NRAS, MAPK1, ARAF and ERBB3, are associated with various histiocytoses (Badalian-Very et al., 2010; Emile et al., 2013, 2014; Brown et al., 2014; Chakraborty et al., 2014; Arceci, 2014); the V600E BRAF mutation is present in as many as 40-60% of patients with systemic histiocytosis, such as Langerhans Cell Histiocytosis (LCH).

A member of the serine/threonine kinase RAF family, the BRAF protein is part of the RAS-RAF-MEK-MAPK signaling pathway that plays a major role in regulating cell survival, proliferation and differentiation (Keshet and Seger, 2010). BRAF mutations constitutively activate the MEK-ERK pathway, leading to enhanced cell proliferation, survival and ultimately, neoplastic transformation (Wellbrock and Hurlstone, 2010; Niault and Baccarini, 2010). In one study, all BRAF mutated LCH cases carried the V600E phospho-mimetic substitution which occurs within the BRAF activation segment and markedly enhances its kinase activity in a constitutive manner (Wan et al., 2004).

Preliminary results suggest that patients with histiocytosis and BRAF mutations can benefit from targeted inhibition of the BRAF protein with BRAF inhibitors (Haroche et al., 2013). Unfortunately, archival tissue often does not provide an adequate amount of DNA for molecular testing. This creates a major hurdle for further implementation of personalized therapies into the ECD therapeutic armamentarium since BRAF inhibitors in general can be effective in patients with BRAF mutations but detrimental in patients without them (Hatzivassiliou et al., 2010). Therefore, novel technologies allowing mutation analysis for ECD and other histiocytosis conditions to be performed using alternative sources of biologic material are needed. The present invention addresses that need.

### BRIEF SUMMARY OF THE INVENTION

The present invention is based on the discovery that gene mutations associated with histiocytosis are present in cell-free DNA in bodily fluids, and that the presence of those gene mutations can be monitored over time to follow the course of the disease.

Thus, in some embodiments, a method of detecting a mutation in a histiocytosis patient is provided. The method comprises (a) obtaining a sample of a bodily fluid from the patient; and (b) testing the sample for the presence of a mutation in a gene in the RAS-RAF-MEK-ERK or the RAS-PI3K-AKT pathway in cell free DNA (cfDNA) in the bodily fluid.

In other embodiments, a method of monitoring disease course of a histiocytosis in a patient having a mutation in a gene in the RAS-RAF-MEK-ERK or the RAS-PI3K-AKT pathway is provided. The method comprises

(a) obtaining a first DNA-containing sample from the patient;

(b) quantifying the mutation and its corresponding wildtype sequence in the first sample at a first time point; and

(c) repeating (a) and (b) at a second time point with a second sample,

wherein an increase in the quantity of the mutation relative to its corresponding wildtype between the first and second time point indicates that the histiocytosis is progressing, and a decrease in the quantity of the mutation relative to its corresponding wildtype indicates that the histiocytosis is remitting.

Also provided is a method of selecting and/or applying treatment or therapy for a histiocytosis patient. The method comprises detecting a mutation in the patient or monitoring disease course of the patient's histiocytosis by the above methods; and selecting and/or applying a treatment or therapy based on the detecting or monitoring.

Further provided is a method of treating a patient having a histiocytosis. The method comprises

(a) testing for the quantity of a mutation in a gene in the RAS-RAF-MEK-ERK or the RAS-PI3K-AKT pathway and a corresponding wildtype sequence in DNA-containing samples taken from the patient at a plurality of time points;

(b) determining whether the quantity of the mutation relative to its corresponding wildtype sequence increased from an earlier time point to a later time point; and

(c) selecting and/or applying a treatment or therapy based on the determining.

### BRIEF DESCRIPTION OF THE DRAWINGS

FIG. 1 illustrates an exemplary two-step assay design for a 28-30 by footprint in the target gene sequence.

FIG. 2 are graphs of experimental results showing positive and negative controls for the identification of a BRAF V600E mutation.

FIG. 3 is a graph showing results from an ECD afflicted patient during treatment with vemurafenib. Sensitivity to the therapy is observed.

FIG. 4 is a graph showing results from an ECD afflicted patient treated with anakinra (trade name Kineret) followed by termination of treatment and then administration of treatment with vemurafenib.

FIG. 5 is a graph showing results from an ECD afflicted patient during treatment with anakinra. Sensitivity to the therapy is observed.

FIG. 6 is graphs showing that BRAF sampling in urinary cfDNA detection of ECD improves chances of success over biopsies due to the high correlation in performance between urinary cfDNA and both tissue samples and plasma from blood samples. The pie graphs on the left of FIG. 6 shows resolution, by use of the methods disclosed herein, of tissue biopsy samples (n=6 BRAF wildtype; n=14 BRAF V600E mutant and n=10 indeterminate genotype) by use of urinary cfDNA into n=18 BRAF wildtype and n=12 BRAF V600E mutant. The remainder of FIG. 6 shows a correlation between urinary cfDNA and plasma cfDNA in detecting BRAF wildtype, mutant, and unknown genotypes when using the methods disclosed herein.

FIG. 7 is graphs showing BRAF V600E mutant allele burden in cell-free DNA (cfDNA) of urine and plasma from treatment naïve patients based on BRAF V600E tissue genotype result. Panel A shows the ratio of BRAF V600E:BRAF wildtype in urinary cfDNA of patients based on BRAF mutational status of histiocytosis tissue biopsy (BRAF V600E mutant, BRAF wildtype, or BRAF mutational status unknown). Panel B shows the ratio of BRAF V600E:BRAF wildtype in plasma cell-free DNA of patients based on BRAF mutational status of histiocytosis tissue biopsy. Each point represents a single test result from evaluation before initiation of any therapy. The dashed line indicates the cutpoint indicating the presence of the BRAF V600E mutation.

FIG. 8 is graphs showing BRAF V600E mutant allele burden in cell-free DNA (cfDNA) of urine and plasma based on BRAF V600E tissue genotype result. Panel A is pie chart representations of BRAF V600E mutational genotypes as determined by initial tissue biopsy (left) or urinary cfDNA analysis (right). Results were recorded as BRAF V600E mutant (light shade), BRAF V600E wildtype (white), or result indeterminate (dark shade). Panel B shows the ratio of BRAF V600E:BRAF wildtype in urinary cfDNA of patients based on BRAF mutational status as determined from tissue biopsy (BRAF V600E mutant, BRAF wildtype, or BRAF mutational status unknown). Panel C shows the ratio of BRAF V600E:BRAF wildtype in plasma cfDNA of patients based on BRAF mutational status as determined from tissue biopsy. Each point represents a single test result from the initial assessment of BRAF V600E:BRAF wildtype allelic ratio in cfDNA. Dotted points represent samples collected during RAF inhibitor therapy. The dashed line indicates the cutpoint indicating the presence of the BRAFV600E mutation.

FIG. 9 is graphs showing the effect of therapy on BRAF V600E mutant allele burden in cell-free DNA (cfDNA) of systemic histiocytosis patients. Panel A shows a comparison of BRAF V600E allele burden in treatment naïve urine samples compared with urinary samples acquired anytime during therapy. Panel B shows the effect of RAF inhibitors on cfDNA BRAF V600E mutant allele burden in 7 consecutive patients treated with RAF inhibitors. The initial sample in each patient is prior to initiation of therapy. The dashed line indicates the cutpoint indicating the presence of the BRAFV600E mutation.

FIG. 10 shows graphs and clinical imaging results demonstrating dynamic monitoring of serial urinary cell-free DNA (cfDNA) BRAF V600E mutant allele burden in systemic histiocytosis patients. Panel A shows gadolinium-enhanced T1 MRI images of ECD involvement of brain (arrows), and ¹⁸F-FDG-PET images of disease in the right atrium (asterisk) and testes (asterisk), pre-dabrafenib and after 2 months of dabrafenib. Panel B shows urinary BRAF V600E cfDNA results throughout this same patient's therapy. Panel C shows urinary BRAF V600E cfDNA results of an ECD patient treated with anakinra followed by a period of treatment cessation and then initiation of vemurafenib. Panel D shows maximal intensity projection (MIP) images of ¹⁸F-FDG-PET scan images demonstrating tibial infiltration by ECD pre-vemurafenib, following 10 weeks of vemurafenib, and then 16 weeks after vemurafenib discontinuation in an ECD patient (top) with accompanying urinary cfDNA results for each time point (below).

FIG. 11 shows radiographic, histologic, and molecular evaluation of KRAS G12S mutant patient with Erdheim-Chester Disease (ECD). Panels A and B show an ¹⁸F-FDG-PET scan result of an ECD patient who was BRAF V600E wildtype by tissue biopsy and urinary and plasma cell-free DNA analysis revealing PET avidity of heart (Panel A) and right atrium specifically (Panel B). Panel C shows a hematoxylin-eosin stained histological section of cardiac tissue biopsy showing a prominent histiocytic infiltrate. Histiocytes have abundant pale staining, finely granular cytoplasm. Panel D shows a screen shot using Integrated Genomics Viewer (IGV) demonstrates the presence of KRAS G12S mutation in DNA from a histiocyte tissue biopsy. Panel E shows next-generation sequencing of PCR enriched amplicon from urine and plasma derived cfDNA confirming KRAS G12S mutation.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

As used herein, the term “sample” refers to anything which may contain an analyte for which an analyte assay is desired. In many cases, the analyte is a cf nucleic acid molecule, such as a DNA or cDNA molecule encoding all or part of BRAF. The sample may be a biological sample, such as a biological fluid or a biological tissue. Examples of biological fluids include urine, blood, plasma, serum, saliva, semen, stool, sputum, cerebrospinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, a “patient” includes a mammal. The mammal can be e.g., any mammal, e.g., a human, primate, bird, mouse, rat, fowl, dog, cat, cow, horse, goat, camel, sheep or a pig. In many cases, the mammal is a human being.

The present invention is based in part on the discovery that gene mutations associated with histiocytosis, such as BRAF V600E, is present in cell-free DNA in bodily fluids, and that the presence of those gene mutations can be monitored over time to follow the course of the disease. See Examples below.

Cell-free DNA (cfDNA) is released to the circulation from cells undergoing apoptosis, necroptosis and active secretion and has been identified in the plasma and urine of patients with cancer (De Mattos-Arruda et al., 2013; Crowley et al., 2013). Detecting and quantifying the amount of mutant cfDNA fragments harboring specific mutations can be used as an alternative to tissue testing. Some data suggest that the amount of mutant DNA correlates with tumor burden and can be used to identify the emergence of resistant mutations (Forshew et al., 2012; Murtaza et al., 2013; Dawson et al., 2013; Diaz et al., 2012; Misale et al., 2012; Diehl et al., 2008). The concept of mutation testing from urine cfDNA has been assessed in a pilot study in patients with advanced colorectal cancer and other colorectal diseases in which KRAS mutations in urine cfDNA were concordant in 95% of cases with KRAS mutation status in tumor tissue (Su et al., 2008).

Thus, in some embodiments, a method of detecting a mutation in a histiocytosis patient is provided. The method comprises (a) obtaining a sample of a bodily fluid from the patient; and (b) testing the sample for the presence of a mutation in cell free DNA (cfDNA) in the bodily fluid. In some embodiments, the mutation is in a gene in the RAS-RAF-MEK-ERK or the RAS-PI3K-AKT pathway.

Any bodily fluid that would be expected to have cfDNA can be utilized in these methods. Non-limiting examples of a bodily fluid include, but are not limited to, peripheral blood, serum, plasma, urine, lymph fluid, amniotic fluid, and cerebrospinal fluid. In certain particular embodiments, such as those illustrated in the Examples, the bodily fluid is serum, plasma or urine.

It is expected that any mutation, particularly any mutation in a gene in the RAS-RAF-MEK-ERK or the RAS-PI3K-AKT pathway, that leads to enhanced cell proliferation could be utilized in any of the methods disclosed herein. Examples of genes in these pathways having mutations associated with histiocytosis are BRAF, PIK3A, NRAS, MAPK1, ARAF or ERBB3 genes.

In particular embodiments, the mutation is in a BRAF gene. In many cases, the BRAF mutation is a BRAF V600E mutation, in which a glutamic acid (Glu or E) is substituted for a Valine (Val or V) residue at position or amino acid residue 600 of SEQ ID NO: 9. Alternatively, or in addition, the BRAF mutation is a substitution of an adenine (A) for a thymine (T) nucleotide at position 1860 of SEQ ID NO: 1.

Wildtype Homo sapiens v-raf murine sarcoma viral oncogene homolog B1, BRAF, is encoded by the following mRNA sequence (NM_{—}004333, SEQ ID NO:1) (wherein coding sequence is bolded and the coding sequence for amino acid residue 600 is underlined and enlarged):

| | |
|---|---|
| 1 | cgcctccctt ccccctcccc gcccgacagc ggccgctcgg gccccggctc tcggttataa |
| 61 | gatggcggcg ctgagcggtg gcggtggtgg cggcgcggag ccgggccagg ctctgttcaa |
| 121 | cggggacatg gagcccgagg ccggcgccgg cgccggcgcc gcggcctctt cggctgcgga |
| 181 | ccctgccatt ccggaggagg tgtggaatat caaacaaatg attaagttga cacaggaaca |
| 241 | tatagaggcc ctattggaca aatttggtgg ggagcataat ccaccatcaa tatatctgga |
| 301 | ggcctatgaa gaatacacca gcaagctaga tgcactccaa caaagagaac aacagttatt |
| 361 | ggaatctctg gggaacggaa ctgatttttc tgtttctagc tctgcatcaa tggataccgt |
| 421 | tacatcttct tcctcttcta gcctttcagt gctaccttca tctctttcag tttttcaaaa |
| 481 | tcccacagat gtggcacgga gcaaccccaa gtcaccacaa aaacctatcg ttagagtctt |
| 541 | cctgcccaac aaacagagga cagtggtacc tgcaaggtgt ggagttacag tccgagacag |
| 601 | tctaaagaaa gcactgatga tgagaggtct aatcccagag tgctgtgctg tttacagaat |
| 661 | tcaggatgga gagaagaaac caattggttg ggacactgat atttcctggc ttactggaga |
| 721 | agaattgcat gtggaagtgt tggagaatgt tccacttaca acacacaact ttgtacgaaa |
| 781 | aacgtttttc accttagcat tttgtgactt ttgtcgaaag ctgcttttcc agggtttccg |
| 841 | ctgtcaaaca tgtggttata aatttcacca gcgttgtagt acagaagttc cactgatgtg |
| 901 | tgttaattat gaccaacttg atttgctgtt tgtctccaag ttctttgaac accacccaat |
| 961 | accacaggaa gaggcgtcct tagcagagac tgccctaaca tctggatcat ccccttccgc |
| 1021 | acccgcctcg gactctattg ggccccaaat tctcaccagt ccgtctcctt caaaatccat |
| 1081 | tccaattcca cagcccttcc gaccagcaga tgaagatcat cgaaatcaat ttgggcaacg |
| 1141 | agaccgatcc tcatcagctc ccaatgtgca tataaacaca atagaacctg tcaatattga |
| 1201 | tgacttgatt agagaccaag gatttcgtgg tgatggagga tcaaccacag gtttgtctgc |
| 1261 | taccccccct gcctcattac ctggctcact aactaacgtg aaagccttac agaaatctcc |
| 1321 | aggacctcag cgagaaagga agtcatcttc atcctcagaa gacaggaatc gaatgaaaac |
| 1381 | acttggtaga cgggactcga gtgatgattg ggagattcct gatgggcaga ttacagtggg |
| 1441 | acaaagaatt ggatctggat catttggaac agtctacaag ggaaagtggc atggtgatgt |
| 1501 | ggcagtgaaa atgttgaatg tgacagcacc tacacctcag cagttacaag ccttcaaaaa |
| 1561 | tgaagtagga gtactcagga aaacacgaca tgtgaatatc ctactcttca tgggctattc |
| 1621 | cacaaagcca caactggcta ttgttaccca gtggtgtgag ggctccagct tgtatcacca |
| 1681 | tctccatatc attgagacca aatttgagat gatcaaactt atagatattg cacgacagac |
| 1741 | tgcacagggc atggattact tacacgccaa gtcaatcatc cacagagacc tcaagagtaa |
| 1801 | taatatattt cttcatgaag acctcacagt aaaaataggt gattttggtc tagctacagt |
| 1861 | gaaatctcga tggagtgggt cccatcagtt tgaacagttg tctggatcca ttttgtggat |
| 1921 | ggcaccagaa gtcatcagaa tgcaagataa aaatccatac agctttcagt cagatgtata |
| 1981 | tgcatttgga attgttctgt atgaattgat gactggacag ttaccttatt caaacatcaa |
| 2041 | caacagggac cagataattt ttatggtggg acgaggatac ctgtctccag atctcagtaa |
| 2101 | ggtacggagt aactgtccaa aagccatgaa gagattaatg gcagagtgcc tcaaaaagaa |
| 2161 | aagagatgag agaccactct ttccccaaat tctcgcctct attgagctgc tggcccgctc |
| 2221 | attgccaaaa attcaccgca gtgcatcaga accctccttg aatcgggctg gtttccaaac |
| 2281 | agaggatttt agtctatatg cttgtgcttc tccaaaaaca cccatccagg cagggggata |
| 2341 | tggtgcgttt cctgtccact gaaacaaatg agtgagagag ttcaggagag tagcaacaaa |
| 2401 | aggaaaataa atgaacatat gtttgcttat atgttaaatt gaataaaata ctctcttttt |
| 2461 | ttttaaggtg aaccaaagaa cacttgtgtg gttaaagact agatataatt tttccccaaa |
| 2521 | ctaaaattta tacttaacat tggattttta acatccaagg gttaaaatac atagacattg |
| 2581 | ctaaaaattg gcagagcctc ttctagaggc tttactttct gttccgggtt tgtatcattc |
| 2641 | acttggttat tttaagtagt aaacttcagt ttctcatgca acttttgttg ccagctatca |
| 2701 | catgtccact agggactcca gaagaagacc ctacctatgc ctgtgtttgc aggtgagaag |
| 2761 | ttggcagtcg gttagcctgg gttagataag gcaaactgaa cagatctaat ttaggaagtc |
| 2821 | agtagaattt aataattcta ttattattct taataatttt tctataacta tttcttttta |
| 2881 | taacaatttg gaaaatgtgg atgtctttta tttccttgaa gcaataaact aagtttcttt |
| 2941 | taaaaa |

Wildtype Homo sapiens v-raf murine sarcoma viral oncogene homolog B1, BRAF, is encoded by the following amino acid sequence (NP_{—}004324, SEQ ID NO: 2) (wherein amino acid residue 600 is bolded and underlined and enlarged):

| | |
|---|---|
| 1 | maalsggggg gaepgqalfn gdmepeagag agaaassaad paipeevwni kqmikltqeh |
| 61 | iealldkfgg ehnppsiyle ayeeytskld alqqreqqll eslgngtdfs vsssasmdtv |
| 121 | tsssssslsv lpsslsvfqn ptdvarsnpk spqkpivrvf lpnkqrtvvp arcgvtvrds |
| 181 | lkkalmmrgl ipeccavyri qdgekkpigw dtdiswltge elhvevlenv pltthnfvrk |
| 241 | tfftlafcdf crkllfqgfr cqtcgykfhq rcstevplmc vnydqldllf vskffehhpi |
| 301 | pqeeaslaet altsgsspsa pasdsigpqi ltspspsksi pipqpfrpad edhrnqfgqr |
| 361 | drsssapnvh intiepvnid dlirdqgfrg dggsttglsa tppaslpgsl tnvkalqksp |
| 421 | gpqrerksss ssedrnrmkt lgrrdssddw eipdgqitvg qrigsgsfgt vykgkwhgdv |
| 481 | avkmlnvtap tpqqlqafkn evgvlrktrh vnillfmgys tkpqlaivtq wcegsslyhh |
| 541 | lhiietkfem iklidiarqt aqgmdylhak siihrdlksn niflhedltv kigdfglatv |
| 601 | ksrwsgshqf eqlsgsilwm apevirmqdk npysfqsdvy afgivlyelm tgqlpysnin |
| 661 | nrdqiifmvg rgylspdlsk vrsncpkamk rlmaeclkkk rderplfpqi lasiellars |
| 721 | lpkihrsase pslnragfqt edfslyacas pktpiqaggy gafpvh |

Any of the methods described herein can be utilized with patients having any histiocytosis that is associated with a mutation, since any mutation associated with a histiocytosis would be expected to be represented in bodily fluids and detectable by the methods described herein, as exemplified in the Examples with the BRAF and KRAS mutations. In various embodiments, the mutation is in a gene in the RAS-RAF-MEK-ERK or the RAS-PI3K-AKT pathway.

In some of the methods described herein, the mutation is a KRAS mutation, e.g., a G12A, G12C, G12D, G12R, G12S, G12V or G13D mutation. See Example 7.

In various embodiments of the methods described herein, the patients are humans. The patients may be of any age, including, but not limited to infants, toddlers, children, minors, adults, seniors, and elderly individuals. In some embodiments, the histiocytosis is Langerhans Cell Histiocytosis (LCH). In other embodiments, the histiocytosis is non-Langerhans Cell Histiocytosis (nLCH). In some of these embodiments, the nLCH is Erdheim-Chester Disease (ECD). Other non-limiting examples of an nLCH include benign cephalic histiocytosis, generalized eruptive histiocytoma, (giant cell) reticulohistiocytoma, hemophagocytic lymphohistiocytosis (HLH), indeterminate cell histiocytosis, juvenile xanthogranuloma (JXG), Kikuchi disease, multicentric reticulohistiocytosis, necrobiotic xanthogranuloma, Niemann-Pick disease, progressive nodular histiocytoma, Rosai-Dorfman disease, Sea-blue histiocytosis, and xanthoma disseminatum. Other possible examples are interdigitating dendritic sarcoma and histiocytic sarcoma. Thus, the histiocytosis can be cancerous or noncancerous.

The term “LCH” or Langerhans Cell Histiocytosis is intended to encompass the same condition that may be identified by other names, such as Abt-Letterer-Si we disease, Eosinophilic Granuloma, Hand-Schuller-Christian Disease, Letterer-Siwe Disease, and Histiocytosis X.

In any of the methods described herein, the mutation can be determined, or quantified, by any method known in the art. Nonlimiting examples include MALDI-TOF, HR-melting, di-deoxy-sequencing, single-molecule sequencing, use of probes, pyrosequencing, second generation high-throughput sequencing, SSCP, RFLP, dHPLC, CCM, or methods utilizing the polymerase chain reaction (PCR), e.g., digital PCR, quantitative-PCR, or allele-specific PCR (where the primer or probe is complementary to the variable gene sequence). In some embodiments, the PCR is droplet digital PCR, e.g., as described in the Examples. In some of these methods, the mutation is quantified along with the wildtype sequence, to determine the percentage of mutated sequence. In other methods, only the mutation is quantified.

In many embodiments, the nucleic acids are cf DNA (“cfDNA”). In some embodiments, the amplified or detected DNA molecule is genomic DNA. In other embodiments, the amplified or detected molecule is a cDNA. In other embodiments, the nucleic acids is cfRNA or cf mRNA.

The assay may be utilized in quantitative, semi-quantitative, or qualitative modes to monitor molecular changes over time.

In some cases, the method is performed quantitatively, such that the amount of the gene alteration is quantitatively determined and may be quantitatively compared to another measurement. Non-limiting examples of methods for quantitative determinations include quantitative PCR or sequencing.

In other cases, the method is performed semi-quantitatively, such that the amount of the gene alteration may be determined and then compared to another measurement simply to determine a relative increase or decrease relative to each other. In additional cases, the method is performed qualitatively, such that the mutation is determined as detectable or not detectable.

The detection limits for the presence of a gene alteration (mutation) in cf nucleic acids may be determined by assessing data from one or more of negative controls (e.g. from healthy control subjects or verified cell lines) and a plurality of patient samples. Optionally, the limits may be determined based in part on minimizing the percentage of false negatives as being more important than minimizing false positives. One set of non-limiting thresholds for BRAF V600E is defined as less than about 0.05% of the mutation in a sample of cf nucleic acids for a determination of no mutant present or wild-type only; the range of about 0.05% to about 0.107% as “borderline”, and greater than about 0.107% as detected mutation. In other embodiments, and with other mutations, a no-detection designation threshold for the mutation is set at less than about 0.2%, less than about 0.3%, less than about 0.4%, less than about 0.5%, less than about 0.6%, less than about 0.7%, less than about 0.8%, less than about 0.9%, or less than about 1% detection of the mutation relative to a corresponding wildtype sequence. Of course the inclusion of additional patient samples may result in the determination of different threshold values for each category, or alternatively the elimination of the “borderline” category. The desired amount of false negatives to false positives will also have an effect on the threshold value.

In some embodiments, the patient has not previously undergone testing for a mutation in a gene, e.g., in the RAS-RAF-MEK-ERK or the RAS-PI3K-AKT pathway. In those situations, the method are used to determine whether a specific mutation is involved in the histiocytosis, and whether a medicament that targets the product of the gene having the mutation could be effective. For example, where a BRAF V600E mutation is present, the patient might be treated with a BRAF inhibitor such as vemurafenib, sorafenib or dabrafenib.

Other known treatments for histiocytosis include anakinra (Kineret), a recombinant form of interleukin-1 receptor antagonist (RA), anthracyclines, cladribine, etoposide, vinblastine, alkylating agents, antimetabolites, vinca alkaloids, immunotherapy (alpha interferon), systemic corticosteroids, immunosuppressants, methotrexate, tamoxifen, imatinib (Gleevec®), infliximab, tocilumab (Actemra®), surgical removal or reduction of any mass which has formed, radiation treatment, antibiotics, and modafinil (Provigil®) and other chemotherapy. Of course a skilled clinician is aware of the recognized and approved treatments and therapies for various forms of histiocytosis, and so the maintenance of, or change in, treatment or therapy may be within those that are known.

In some embodiments, the patient has been previously tested and a mutation determined, and the subsequent tests are to evaluate the course of the disease and/or the effectiveness of treatment. In some cases, the detecting may identify the non-responsiveness to a treatment or therapy, and the selecting and/or applying comprises a different treatment or therapy. In other cases, the detecting may identify the responsiveness to a treatment or therapy, and the selecting and/or applying comprises continuation of the same treatment or therapy.

Thus, the present invention is also directed to a method of monitoring disease course of a histiocytosis in a patient having a mutation in a gene in the RAS-RAF-MEK-ERK or the RAS-PI3K-AKT pathway is provided. The method comprises

(a) obtaining a first DNA-containing sample from the patient;

(b) quantifying the mutation and its corresponding wildtype sequence in the first sample at a first time point; and

(c) repeating (a) and (b) at a second time point with a second sample. In these methods an increase in the quantity of the mutation relative to its corresponding wildtype between the first and second time point indicates that the histiocytosis is progressing, and a decrease in the quantity of the mutation relative to its corresponding wildtype indicates that the histiocytosis is remitting. The methods can be repeated once or more times to provide measurements over time. In some cases, the methods are repeated two or more times, three or more times, four or more times, five or more times, or six or more times. The repetition of the methods may be at regular intervals, or at irregular intervals. Non-limiting examples of intervals include biweekly and monthly.

In some aspects, the monitoring of the mutation is accompanied by a determining the disease burden, e.g., by radiography, computed tomography (CT) scanning, positron emission tomography (PET), or PET/CT scanning, and comparing the determined amount of mutation to the disease burden. This is useful to determine whether, or confirm that the mutation being monitored is actually the driver of the disease.

In other aspects, the determined amount of mutation is not compared to disease burden, either at one, more than one, or all the mutation monitoring times. Given the reliability of the mutation monitoring procedures described herein, a disease burden assessment need not be made at each time point, thus saving the patient a disease burden assessment.

Thus, these methods may be used to confirm the maintenance of a disclosed treatment or therapy against histiocytosis, or to change the treatment or therapy against the disease. Within the scope of changing treatment or therapy, the disclosure includes increasing the treatment or therapy; reducing the treatment or therapy, optionally to the point of terminating the treatment or therapy; terminating the treatment or therapy with the start of another treatment or therapy; and adjusting the treatment or therapy as non-limiting examples. Non-limiting examples of adjusting the treatment or therapy include reducing or increasing the therapy, optionally in combination with one or more additional treatments or therapies; or maintaining the treatment or therapy while adding one or more additional treatments or therapies.

In some cases, the observation of cell-free (cf) nucleic acids identifies an increase in the levels of cf nucleic acids containing the mutation following the start of a treatment or therapy. Following the increase, the observation may reach an inflection point, where the levels decrease, or continue to increase. The presence of an inflection point may be used to determine responsiveness to the treatment or therapy, which may be maintained or reduced. A continuing decrease in the levels to be the same as, or lower than, the levels before the start of treatment of therapy is a further confirmation of responsiveness.

The absence of an inflection point indicates resistance to the treatment or therapy and so may be followed by terminating administration of the treatment or therapy, or administering at least one additional treatment or therapy against the disease or disorder to the patient, reducing the treatment of the subject with the treatment or therapy and administering at least one additional treatment or therapy against the disease or disorder to the subject.

In other cases, and following an inflection point and a decrease in levels, an additional inflection point may be observed. This may indicate the development of resistance to the treatment or therapy and be followed by terminating administration of the treatment or therapy, or administering at least one additional treatment or therapy against the disease or disorder to the subject, or reducing the treatment of the subject with the therapy and administering at least one additional therapy against the disease or disorder to the subject.

In these methods, the samples can be tissue samples or bodily fluid samples. Any tissue sample that provides sufficient nucleic acids to determine the presence of the mutation may be utilized. In some embodiments, the tissue sample is from abnormal tissue associated with the histiocytosis, such as from a lesion or tumor. The tissue can be fresh, freshly frozen, or fixed, such as formalin-fixed paraffin-embedded (FFPE) tissues. The sample can be obtained by any means, for example via a surgical procedure, such as a biopsy, or by a less invasive method, including, but not limited to, abrasion or fine needle aspiration.

Also provided is a method of selecting and/or applying treatment or therapy for a histiocytosis patient. The method comprises detecting a mutation in the patient or monitoring disease course of the patient's histiocytosis by the above methods; and selecting and/or applying a treatment or therapy based on the detecting or monitoring. The various elements of these embodiments are discussed above.

Further provided is a method of treating a patient having a histiocytosis. The method comprises

(a) testing for the quantity of a mutation in a gene in the RAS-RAF-MEK-ERK or the RAS-PI3K-AKT pathway and a corresponding wildtype sequence in DNA-containing samples taken from the patient at a plurality of time points;

(b) determining whether the quantity of the mutation relative to its corresponding wildtype sequence increased from an earlier time point to a later time point; and

(c) selecting and/or applying a treatment or therapy based on the determining.

In some of these embodiments, the patient had not previously undergone testing for the mutation, and a determination that a mutation is present is followed up by additional monitoring, either with or without treatment. In other embodiments, prior to the testing, the patient was treated with a medicament that targets the product of the gene having the mutation.

Also provided herein is a kit for performing the above methods. The kit may include a specific binding agent that selectively binds to a BRAF mutation, and instructions for carrying out any of the method as described herein.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Examples

### Example 1

### Materials and Methods

The following methods described herein were utilized in the examples that follow.

### Patient Urine Samples

Five patients with Erdheim-Chester disease (histiocytic disorder with high prevalence for BRAF mutations) with different tissue involvement were prospectively enrolled. Longitudinal analysis of BRAF V600E in one Erdheim-Chester disease patient was performed by testing serially collected urine.

Separate samples were obtained from patients treated with anakinra for analysis.

### Two-Step Assay Design

A two-step assay design was developed for a 28-30 basepair footprint in the target mutant gene sequence. This assay design (and other assays known in the art) is useful for amplifying any size sequence in various tissues or bodily fluids, for example less than 400, less than 300, less than 200, less than 150 bp, less than 100 bp, less than 50 bp, less than 40 bp, less than 35 bp, or less than 30 bp.

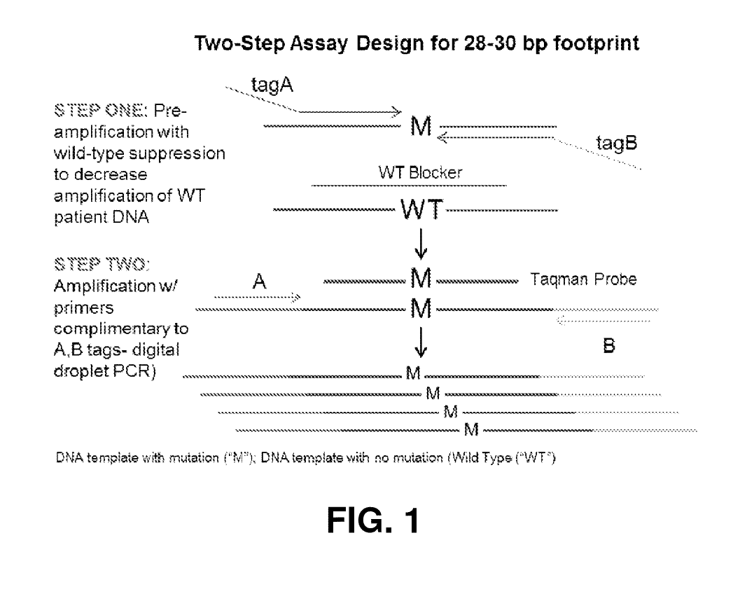

FIG. 1 summarizes the assay design, which includes a first pre-amplification step to increase the number of copies of a target mutant gene sequence relative to wild-type gene sequences that are present in the sample. The pre-amplification is conducted in the presence of a wild-type (non-mutant) suppressing “WT blocker” oligonucleotide that is complementary to the wild-type sequence (but not the mutant sequence) to decrease amplification of wild-type DNA. The pre-amplification is performed with primers that include adapters (or “tags”) at the 5′ end to facilitate amplification in the second step.

The second step is additional amplification with primers complementary to the tags on the ends of the primers used in the first step and a TaqMan (reporter) probe oligonucleotide complementary to the mutant sequence for quantitative, digital droplet PCR (RainDance Technologies, Billerica, Mass.).

### Assay Development

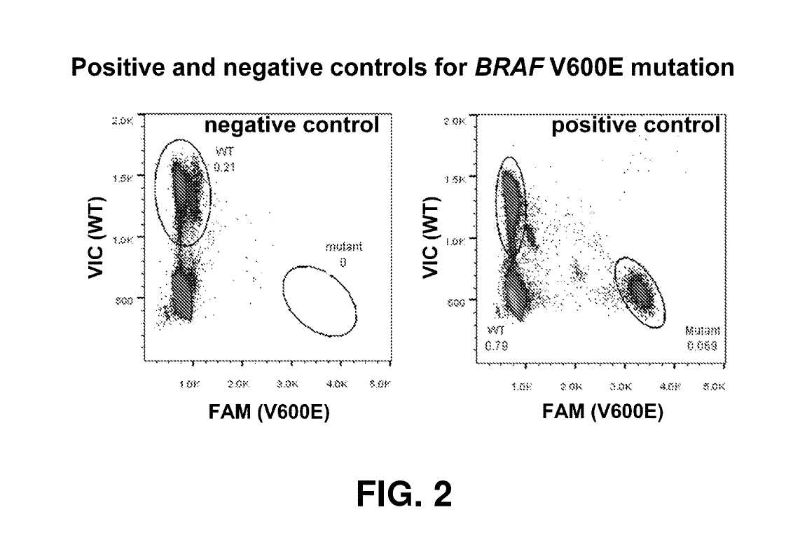

Cell lines with the BRAF V600E mutation were used as positive controls. Cell lines confirmed as wildtype BRAF were used as negative controls. FIG. 2 shows PCR results for positive and negative controls.

Thresholds for mutation detection were determined by assessing data from 50 healthy controls and 39 patient samples using a classification tree. Minimizing the percentage of false negatives was given a higher importance than minimizing false positives.

A set of non-limiting thresholds for BRAF V600E were defined: <0.05% as no detection or wild-type; the range of 0.05% to 0.107% as “borderline”, and >0.107% as detected mutation.

### Example 2

### BRAF V600E Mutations in cfDNA

The sensitivity of the two-step assay was first assessed in urine samples from 5 patients with ECD identified as having a BRAF V600E mutation by a CLIA laboratory. The agreement of CUA V600E to urinary cfDNA V600E mutation and “borderline” is shown in Table 1:

TABLE 1

| | | Urinary cfDNA |
|---|---|---|
| Erdheim Chester Disease | | V600E BRAF |
| Involvement | Tissue (CLIA) | mutation (%) |
| Bones, cardiac, CNS, kidneys | V600E | V600E (129.5) |
| Bones, kidneys | Unknown | Wild-type (0.02) |
| Skin | NTRK1 rearrangement | Wild-type (0.01) |
| Bones | Unknown | V600E (0.16) |
| Bones | Unknown | V600E (4.94) |

### Example 3

### Longitudinal Assessment of cfDNA Mutations

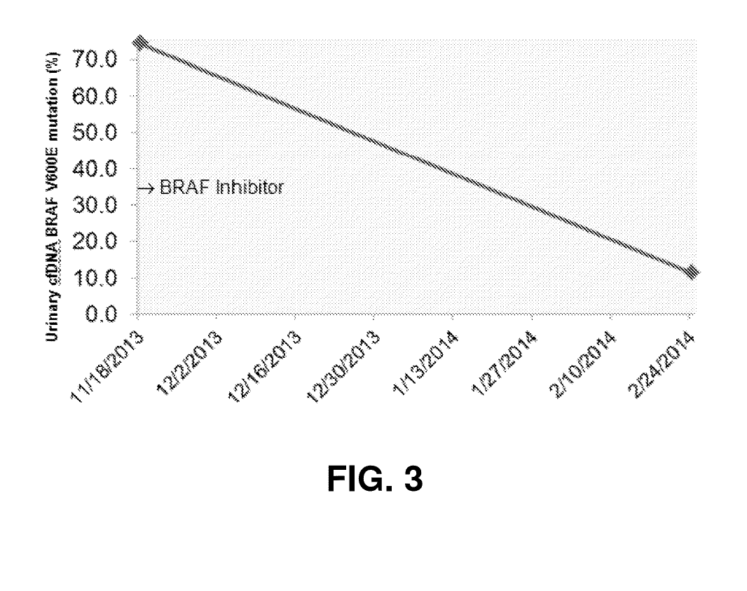

In one patient multiple urine samples obtained over time was assayed as described above. The patient was afflicted with ECD (and treated with the BRAF inhibitor vemurafenib). The results for the patient are shown in FIG. 3, which indicates responsiveness to therapy. Thus the therapy may be maintained or reduced, and monitoring may continue to determine whether ECD recurs.

### Example 4

### Monitoring of Therapeutic Efficacy

In a separate patient multiple urine samples obtained over time was assayed as described above. The patient was afflicted with ECD and first treated with anakinra followed by cessation of the treatment and subsequent treatment with the BRAF inhibitor vemurafenib.

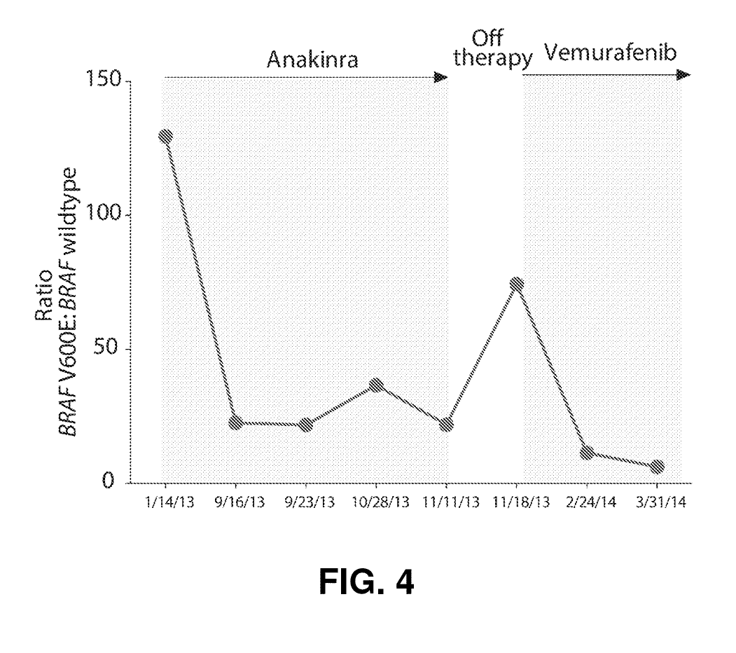

Results from the monitoring of the ratio of the BRAF V600E mutation relative to wildtype BRAF in urine samples are shown in FIG. 4, which indicates responsiveness to anakinra, followed by an increase in BRAF V600E with the cessation of therapy and then responsiveness to vemurafenib.

This demonstrates that the assay indicates responsiveness to two therapeutic agents against ECD without limitation to the mechanism of action of each agent. Additionally, the assay indicates a loss of responsiveness when a therapy ended. This supports the use of the assay to indicate a lack of responsiveness, or lack of disease control, when no therapy, or an ineffective therapy, is used. Moreover, this demonstrates that the assay may be used to monitor a change in therapy as described herein.

### Example 5

### Additional Longitudinal Assessment of cfDNA Mutations

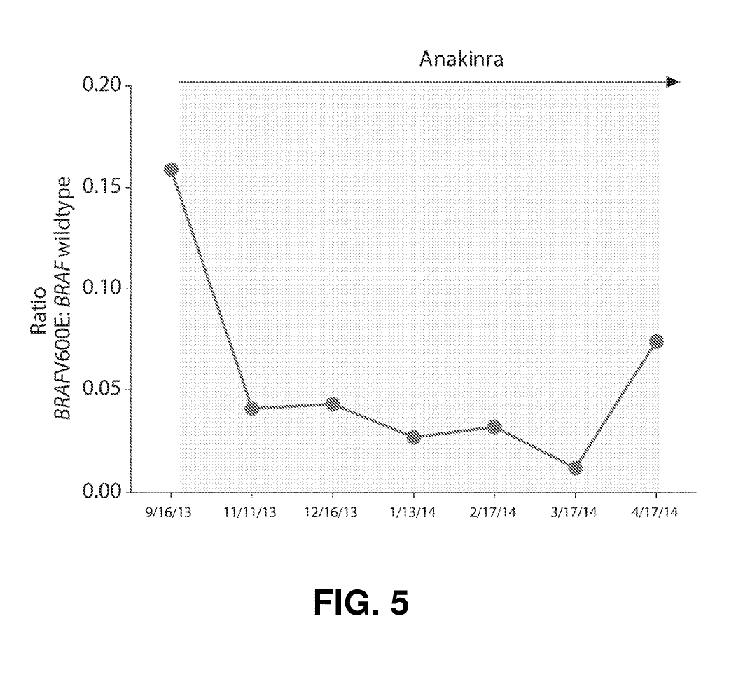

An additional set of patient urine samples obtained over time was assayed as described above. The patient was afflicted with ECD and treated with anakinra. The results for the patient are shown in FIG. 5, which indicates responsiveness to therapy with possible reduction in responsiveness shown by the last time point. Thus the patient may be further monitored with the assay to confirm the reduction in responsiveness to therapy or be switched to a different treatment modality with further monitoring.

### Example 6

### Comparison of Mutational Analysis with Tissue, Serum and Plasma

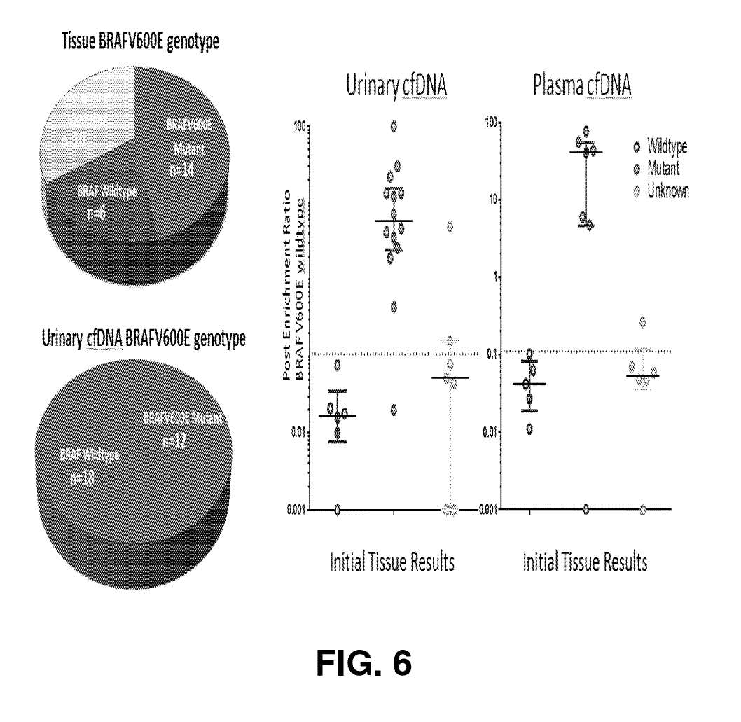

BRAF mutational analysis using tissues, urine and/or plasma of ECD patients were compared. Results are shown in FIG. 6. The pie graphs on the left of FIG. 6 shows resolution, by use of the methods disclosed herein, of tissue biopsy samples (n=6 BRAF wildtype; n=14 BRAF V600E mutant and n=10 indeterminate genotype) by use of urinary cfDNA into n=18 BRAF wildtype and n=12 BRAF V600E mutant. The remainder of FIG. 6 shows a correlation between urinary cfDNA and plasma cfDNA in detecting BRAF wildtype, mutant, and unknown genotypes when using the methods disclosed herein. This shows that urinary cfDNA detection of histiocytosis mutations have a high correlation in performance between urinary cfDNA and both tissue samples and plasma from blood samples.

Additional data from the above studies is provided in Janku et al., 2014.

### Example 7

### Prospective Blinded Study of BRAFV600E Mutation Detection in Cell-Free DNA of Patients with Systemic Histiocytic Disorders

### Example Abstract

Patients with Langerhans Cell Histiocytosis (LCH) and Erdheim-Chester Disease (ECD) have a high frequency of BRAFV600E mutations and respond to RAF inhibitors. However, detection of mutations in tissue biopsies is particularly challenging in histiocytoses due to low tumor content and stromal contamination. A droplet-digital PCR assay for quantitative detection of the BRAFV600E mutation was applied to plasma and urine cell-free (cf)DNA and performed in prospective, blinded study in 30 ECD/LCH patients. There was 100% concordance between tissue and urinary cfDNA genotype in treatment naïve samples. cfDNA analysis facilitated identification of previously undescribed KRASG12S mutant ECD and dynamically tracked disease burden in patients treated with a variety of therapies. These results indicate that cfDNA BRAFV600E mutational analysis in plasma and urine provides a convenient and reliable method of detecting mutational status and can serve as a non-invasive biomarker to monitor response to therapy in LCH and ECD.

### INTRODUCTION

Langerhans Cell Histiocytosis (LCH) and Erdheim-Chester Disease (ECD) are heterogeneous systemic histiocytic disorders characterized by accumulation and infiltration of histiocytes in multiple tissues of the body leading to organ compromise (Janku et al., 2013). Although the underlying etiology of these conditions has long been enigmatic, recent investigations have determined that both LCH and ECD are clonal disorders of myeloid-derived precursor cells with a high frequency of somatic BRAF V600E mutations (40-60% of patients) (Berres et al., 2014; Cangi et al., 2014; Badalian-Very et al., 2010; Arnaud et al., 2012; Satoh et al., 2012; Sahm et al., 2012). Moreover, treatment of BRAF-mutant LCH and ECD patients with the BRAF inhibitor vemurafenib has demonstrated dramatic efficacy revolutionizing the care of these orphan diseases (Haroche et al., 2013).

The above data underline the importance of accurately identifying BRAF mutational status in patients with systemic LCH and ECD (Girchikofsky et al., 2013). Unfortunately, the scant histiocyte content and marked stromal contamination, which are a hallmark of these disorders, make mutation detection in tissue biopsies challenging (Cangi et al., 2014). Moreover, the propensity of histiocytic lesions to involve difficult to biopsy locations such as the brain, orbits, and right atrium frequently necessitates the use of bone biopsies further limiting the availability of suitable tumor material for BRAF genotyping (Girschikofsky et al., 2013). Finally, the infiltrative and multifocal nature of these diseases as well as the absence of a reliable tumor marker has made evaluation of treatment response challenging.

Given these factors, the use of circulating tumor cell-free DNA (cfDNA) to both identify the BRAF V600E mutation and monitor response to therapy represents a potentially transformative development for these orphan diseases. Examples 1-6 above demonstrate that BRAF V600E mutations could be detected in cfDNA (Janku et al., 2014), and the concordance of cfDNA BRAF mutational genotype with tissue mutational status in ECD and LCH. Those Examples also demonstrate the ability of quantitative urine and plasma cfDNA analysis to detect dynamic changes in BRAF V600E mutation burden during treatment of disease. Use of urine as a source of cfDNA offers significant advantages in sample stability and ease of serial collection.

To reinforce the validity and clinical utility of plasma and urine cfDNA BRAF testing in LCH and ECD patients established in Examples 1-6, a blinded, prospective multicenter study in these disorders was performed.

### Patients and Methods

Patients.

Between January 2013 and June 2014, 30 consecutive patients with LCH and ECD seen at Memorial Sloan Kettering Cancer Center (MSKCC) and MD Anderson Cancer Center (MDACC) were enrolled in the study.

Tissue biopsies were performed as part of routine clinical care, with the site of biopsy based on radiographic and/or clinical assessment of disease involvement. 10 mL of blood and between 60-120 mL of urine was collected at each time point. Plasma was separated from blood samples using standard techniques. All samples were de-identified, and operators performing plasma and urine cfDNA analyses were blinded to the tissue genotype and clinical characteristics of all patients.

Institutional Review Boards at both Memorial Sloan Kettering Cancer and MD Anderson Cancer Center approved the study protocol.

Of note, 6 plasma and 6 urinary cfDNA values which were previously reported in a pilot proof-of-concept study 12 are not included in the current study or data analysis.

Tissue Mutational Genotyping.

Initial BRAF tissue mutation testing was performed by a variety of methods as part of routine care in CLIA-certified molecular diagnostic laboratories at MSKCC, MDACC, or the institution from which the patient was initially referred. Tissue with a BRAF V600E mutation identified as part of these analyses was considered positive. For tissue to be considered negative for the BRAF V600E mutation for the purposes of this analysis, it was required to undergo further testing by a high sensitivity assay, either Sanger sequencing with locked nuclear acid (LNA) clamping or next-generation sequencing. Sequencing with LNA was performed according to previously published procedures (Arcila et al., 2011) and had a limit of detection of 0.5% mutant alleles. Massively parallel sequencing was performed by Foundation Medicine Inc. using previously published methodologies (Frampton et al., 2013) with a minimum coverage of 500×. In patients for whom initial diagnostic tissue was insufficient for genotyping, additional biopsies were attempted as deemed appropriate by the treating physician. Patients were considered tumor BRAF indeterminate if they met one of the following criteria: 1) inadequate tumor material for genotyping despite multiple biopsy attempts, 2) declined repeated biopsy for the purpose of genotyping, 3) tissue genotyping was ordered but no result was obtained due to failure of the tumor material to meet technical requirements. Next-generation sequencing of genomic DNA from one BRAF wildtype tumor tissue biopsy was performed on a panel of 30 genes (ASXL1, CBL, CEBPA, DNMT3A, ETV6, EZH2, FLT3, HRAS, IDH1/2, JAK1/2/3, KIT, KRAS, MPL, NPM1, NRAS, PHF6, PTEN, R UNX1, SF3B1, SH2B3, SUZ12, TET1-3, TP53, TYK2, and WT1) by MiSeq at a depth of >500×.

Plasma and Urine cfDNA Extraction and Analyses.

Plasma cfDNA was isolated using the QIAamp Circulating Nucleic Acid Kit (QIAGEN; Germantown, Md.) according to the manufacturer's instructions. Urine cfDNA was isolated as previously described (Janco et al., 2014).

Urine and plasma cfDNA were quantified by a droplet digital PCR (ddPCR; QX-100, BioRad) assay to a 44 bp amplicon of RNase P, a single-copy gene as previously described (Janco et al., 2014). Quantified DNA up to 60 ng was used for mutation detection of BRAF V600E by droplet digital PCR and KRAS mutations at codons 12 and 13 of exon 2 by massively parallel sequencing.

For BRAF V600E mutation detection, a two-step PCR assay targeting a very short (31 bp) amplicon was employed to enhance detection of rare mutant alleles in cfDNA. The first step amplification was done with two primers flanking the BRAF V600E locus, where both primers contain non-complementary 5′ tags that hybridize to second round primers. A complementary blocking oligonucleotide suppressed wildtype BRAF amplification, achieving enrichment of the mutant BRAF V600E sequence in this step. The second step entailed a duplex ddPCR reaction using FAM (BRAF V600E) and VIC (wildtype BRAF) TaqMan probes to enable differentiation of mutant versus wildtype quantification, respectively. The RainDrop ddPCR platform (RainDance; Billerica, Mass.) was used for PCR droplet separation, fluorescent reading, and counting droplets containing mutant sequence, wildtype sequence, or unreacted probe.

For each patient sample, the assay identified BRAF V600E mutation fragments detected as a percentage of detected wildtype BRAF. As previously published, thresholds for the BRAF assay were initially developed by evaluating a training set of urinary cfDNA from BRAF V600E metastatic cancer patients (positives) and healthy volunteers (negatives) using a classification tree that maximized the true positive and true negative rates (Janku et al., 2014; Breiman et al., 1984). Using this training set, a double threshold approach with an indeterminate range between not detected and detected was estimated yielding two threshold values (<0.05=not detected; 0.05-0.107=indeterminate; >0.107 detected) (Janku et al., 2014). For this current study, the assay was simplified to a dichotomous classifier by combining both indeterminate and negative range as ‘not detected’ yielding a single cutoff of <0.107 for not detected and >0.107 as detected. This pre-specified single cutpoint of 0.107 was chosen given that positive and negative BRAF V600E status for ECD patients from a previous study was not within the indeterminate range (Janku et al., 2014). For plasma detection, wildtype BRAF patients with metastatic cancer were used to determine a threshold for detection of BRAF V600E mutations. The BRAF V600E values for this wildtype BRAF population were normally distributed and therefore a pre-specified cutpoint equivalent to three standard deviations (0.021%) above the mean of wildtype BRAF controls (0.031%) or >0.094% mutant to wildtype was considered positive for BRAF V600E12.

For KRAS mutation detection (G12A/C/D/R/S/V, G13D), a two-step PCR assay similar to that described for BRAF V600E was employed with an initial 31 by targeted region, except that during the second round, flanking primers were used to add patient specific barcodes and adaptor sequences necessary for massively parallel DNA sequencing per manufacturer's instructions (MiSeq, Illumina; San Diego, Calif.). Sequence reads were filtered for quality (Q-score>20) and verified as matching the target sequence (no more than 3 mismatches permitted outside the mutation region). For each sample, KRAS mutant sequences were tallied and the percent of mutant was computed. For the KRAS assay, the distribution of background signal in the wildtype population was observed not to conform to a normal distribution. To be consistent with the plasma BRAF assay approach for computing the threshold (mean+3SD), the median and median absolute deviation of a KRAS wildtype population was used to produce a “robust” z-score and a cutoff of greater than 4 z-scores above the median mutant signal count of the population (or >0.0092%) was determined to be a positive result (Malo et al., 2006). This approach is approximately equal to the mean+3SD threshold when the data is normally distributed (data not shown).

Statistical Analysis.

Statistical analyses were performed with GraphPad Prism V5.0 for Macintosh (GraphPad Prism Software, San Diego, Calif.). The Mann-Whitney U test was used to compare BRAF V600E mutant:wildtype ratios determined by cfDNA analysis in patients thought to be BRAF wildtype based on tissue biopsy versus those identified as BRAF V600E mutant based on tissue biopsy. In addition, the Mann-Whitney U test was also used to compare BRAF V600E mutant:wildtype ratios obtained from urinary cfDNA pre-treatment with vemurafenib versus urinary cfDNA BRAF V600E mutant:wildtype ratios obtained following initiation of therapy with vemurafenib. Concordance of tissue, plasma, and urinary assessment of BRAF V600E mutational detection was performed by calculating the kappa coefficient. Correlation of BRAF V600E:BRAF wildtype ratios based on BRAF tissue genotype was performed using Mann-Whitney U test. A two-tailed p-value <0.05 was considered statistically significant.

### Results

Cross-Sectional Analysis.

Data from 30 patients (25 ECD, 5 LCH) were analyzed. Patient and sample characteristics are shown in Table 2. Of these 30 patients, initial tissue BRAFV600E genotyping identified 15 patients to be mutant, 6 patients as wildtype, and 9 as indeterminate. Bone represented the most common anatomic site of attempted tissue acquisition, accounting for 36.7% of biopsies in this cohort (Table 2).

TABLE 2
Patient and Sample Characteristics

| Characteristic | Number (%) | |
|---|---|---|
| Median Age (range) | 56 | (9-75) |
| Sex | | |
| Male | 16 | (53.3%) |
| Female | 14 | (46.7%) |
| Diagnosis | | |
| Erdheim-Chester Disease (ECD) | 25 | |
| Langerhans Cell Histiocytosis (LCH)** | 5 | |
| Sites of tissue biopsy (% of cohort (# of patients)) | | |
| Bone | 36.7% | (11) |
| Abdominal soft tissue (e.g. retroperitoneum) | 26.7% | (8) |
| Skin | 20.0% | (6) |
| Central nervous system | 16.7% | (5) |
| Cardiac tissue | 6.7% | (2) |
| Median Number organ sites involved | | |
| ECD | 3 | (0-11) |
| LCH* | 2 | (1-4) |
| Median Number of Prior Treatments (range)** | 1 | (0-4) |
| Tissue BRAFV600E genotype | | |
| Mutant | 15 | (50%) |
| Wildtype | 6 | (20%) |
| Indeterminate (insufficient tissue or test failure) | 9 | (30%) |
| Median number of urine collections | 2 | (1-8) |
| (per patient, range) | | |
| Median number of plasma collections | 1 | (0-7) |
| (per patient, range) | | |

| | |
|---|---|
| Number of paired urine and plasma collections | 27 |
| Number of patients with initial sample acquired | 26 |
| while untreated | |

Urinary cfDNA analysis for detection of the BRAF V600E mutation was performed on all patients and concordance between cfDNA and tissue DNA mutational results were analyzed. There was 100% concordance between tissue and urinary cfDNA genotype in samples from treatment naïve patients. Urinary BRAF V600E cfDNA values obtained from any time point in therapy identified 16 patients as mutant and 14 as wildtype (Table 3) (kappa=0.88; 95% CI 0.66 to 1.0). This resulted in a sensitivity of urinary cfDNA BRAF V600E detection of 92.9%, a specificity of 100%, a positive predictive value of 100%, and a negative predictive value of 85.7% (all compared to BRAF V600E detection from tissue biopsy). Overall, urinary cfDNA analysis identified 2 patients as being BRAF V600E mutant that were not known to have the BRAF mutation previously. Subsequent tissue biopsy was performed in these patients and identified the BRAF V600E mutation, allowing both patients to enroll in an ongoing phase II study of vemurafenib for BRAF V600E mutant ECD and LCH patients (NCT01524978). Thus, tissue-base genotyping resulted in 21/30 (70%) patients with definitive BRAF status compared to 30/30 (100%) using urinary cfDNA (FIG. 1A).

TABLE 3
Concordance of initial urinary cell-free DNA (cfDNA) assessment of
BRAF V600E mutation with tissue biopsy BRAF V600E result.

| | | Tissue | Tissue biopsy | cfDNA |
|---|---|---|---|---|
| | Tissue biopsy | biopsy | result | results |
| | positive | negative | indeterminate | Total |
| Urinary cfDNA | 14 | 0 | 2 | 16 |
| positive | | | | |
| Urinary cfDNA | 1* | 6 | 7 | 14 |
| negative | | | | |
| Tissue Biopsy | 15 | 6 | 9 | |
| Results Total | | | | |

*Sample obtained during RAF inhibitor therapy.

Urinary cfDNA analysis failed to detect the BRAF V600E mutation in 1/15 (6.7%) patient positive by tissue biopsy. Of note, the urine and plasma utilized for cfDNA analysis in that case were sampled while the patient was in active treatment with a BRAF inhibitor with ongoing reduction in disease burden, whereas the tissue genotyping was performed prior to treatment.

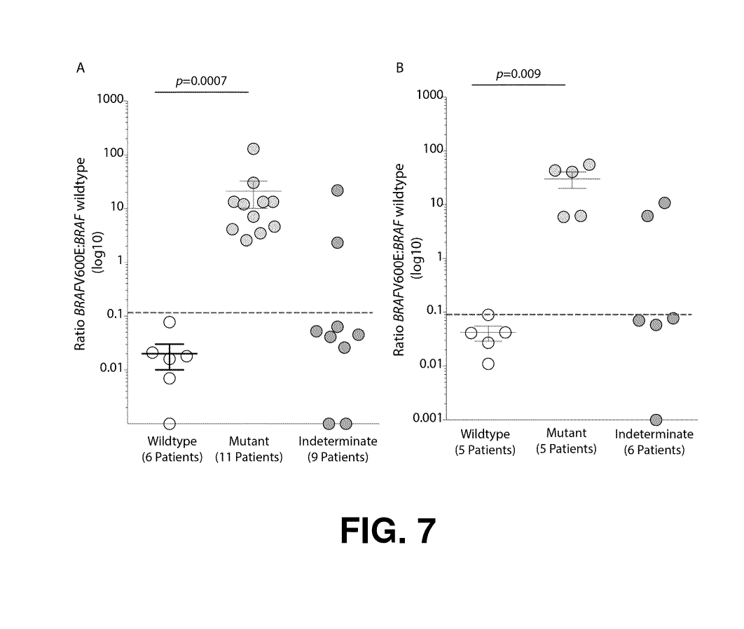

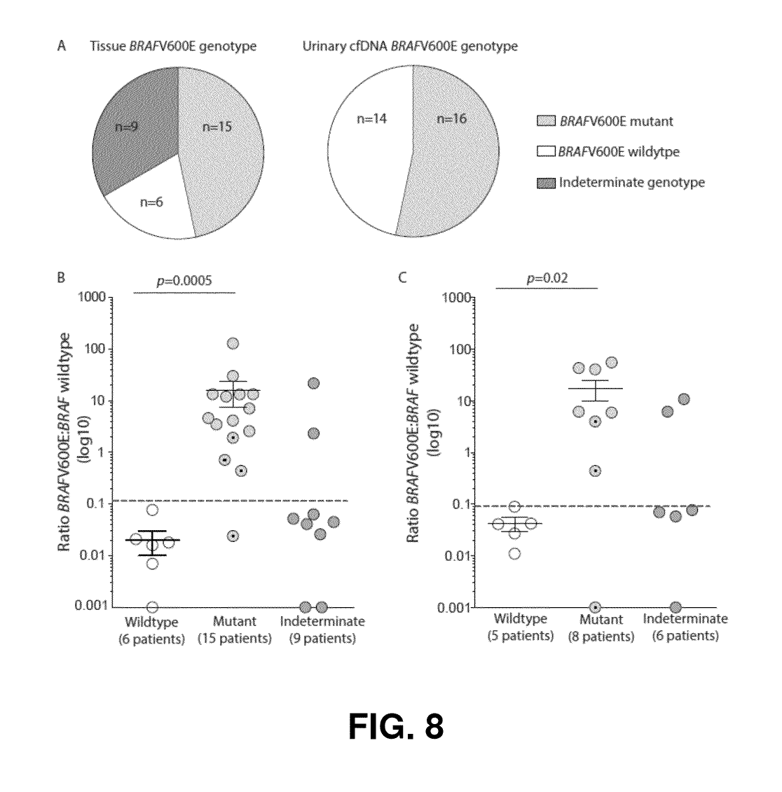

Plasma cfDNA and urinary cfDNA were obtained at the same time point in 19/30 (63.3%) patients. Results from plasma cfDNA for identifying the BRAF V600E mutation were comparable to urinary cfDNA results (Table 4 and FIG. 7). Plasma cfDNA analysis identified 9 patients as mutant and 10 as wildtype. BRAF genotype as determined by urinary and plasma cfDNA assay was concordant for all samples from the 19 patients with both tests (n=26 tests), except one (which was obtained from a patient during RAF inhibitor therapy; 96% concordance). Quantitative BRAF V600E mutant:wildtype ratio was significantly higher in the cfDNA from plasma as well as urine in those patients whose tissue was BRAF V600E versus wildtype (p=0.0005 and 0.002, respectively; FIG. 8B-8C).

TABLE 4
Concordance of initial plasma cell-free DNA (cfDNA) assessment of
BRAF V600E mutations with tissue biopsy BRAF V600E result.

| | | Tissue | Tissue biopsy | cfDNA |
|---|---|---|---|---|
| | Tissue biopsy | biopsy | result | results |
| | positive | negative | indeterminate | Total |
| Plasma cfDNA | 7 | 0 | 2 | 9 |
| positive | | | | |
| Plasma | 1* | 5 | 4 | 10 |
| cfDNA | | | | |
| negative | | | | |
| Tissue Biopsy | 8 | 5 | 6 | |
| Results Total | | | | |

Longitudinal Assessment of BRAF V600E cfDNA Burden.

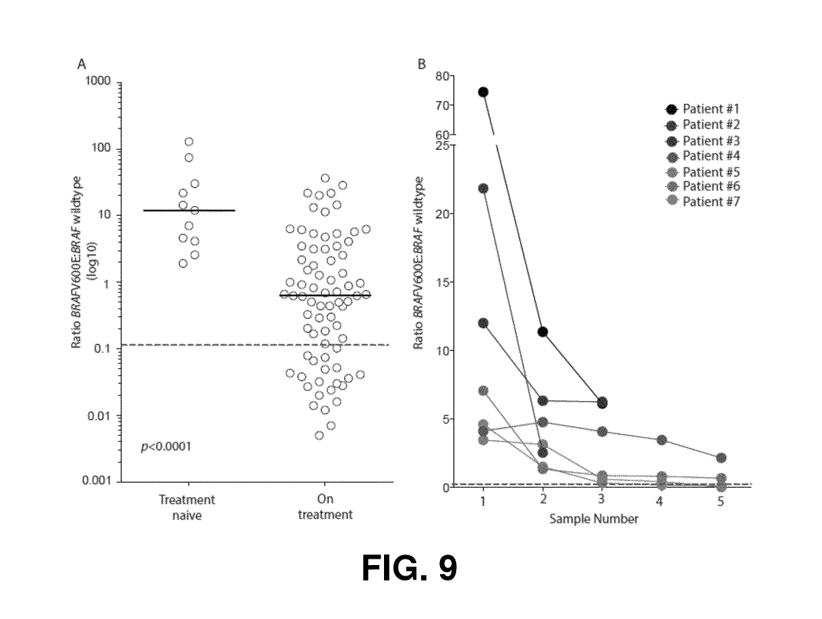

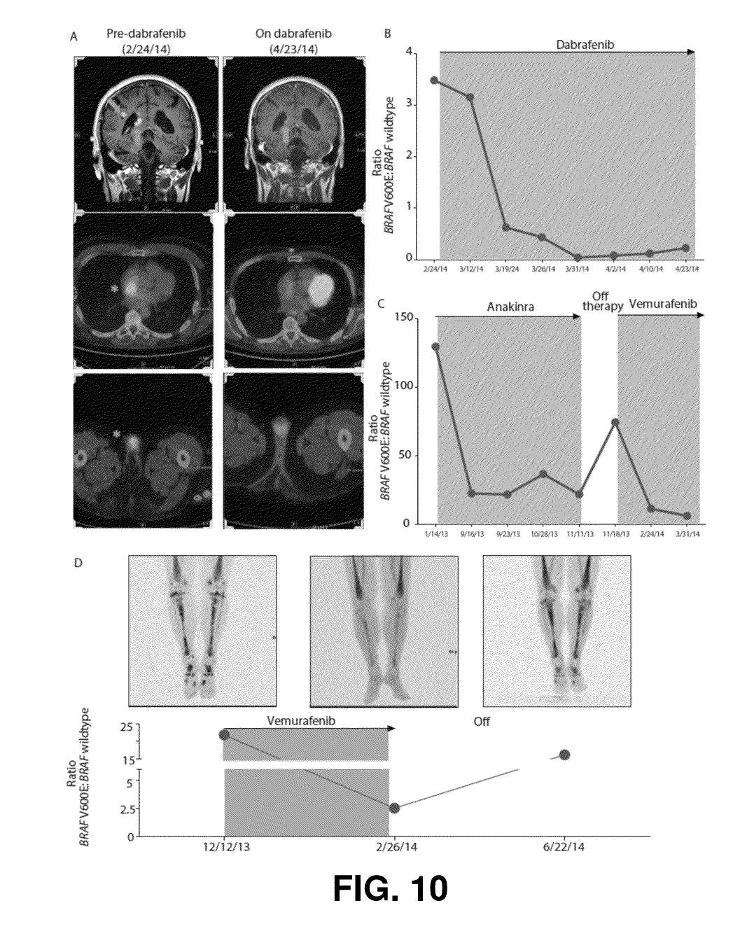

Comparing cfDNA BRAF V600E:BRAF wildtype ratios of pre-treatment versus BRAF inhibitor-treated BRAF V600E mutant patients, a significant decrease in the BRAF V600E:BRAF wildtype ratio was seen with therapy (p<0.0001; FIG. 9A). Serial samples on 13 BRAF V600E mutant patients were available, 10 of which were treated with a BRAF inhibitor. In all patients treated with a BRAF inhibitor, serial urinary cfDNA analysis revealed progressive decrements in the BRAF V600E allele burden (FIG. 9B). Weekly serial urinary cfDNA analysis throughout the course of BRAF inhibitor therapy revealed that the decline in mutant cfDNA burden in response to BRAF inhibitors was consistent with radiographic disease improvement (FIG. 10). Moreover, in at least one patient where successful RAF inhibitor therapy was discontinued for toxicity, urinary cfDNA BRAFV600E burden increased after vemurafenib discontinuation which mirrored radiographic evidence of disease recurrence (FIG. 10D).

Serial cfDNA BRAF V600E burden was also assessed in 2 patients treated with anakinra, an IL-1 receptor antagonist commonly used as an off-label treatment for ECD17. Interestingly, treatment with anakinra also reduced the BRAF V600E mutant allele burden (FIG. 10C). Anakinra was subsequently discontinued in one patient and within 7 days the urinary cfDNA BRAF V600E allele burden increased. Vemurafenib was then initiated in this patient and once again BRAF V600E allele burden as assessed in cfDNA decreased within 2 weeks of BRAF inhibitor therapy.

Identification of a KRAS Mutation in a BRAF V600 Wildtype Patient.

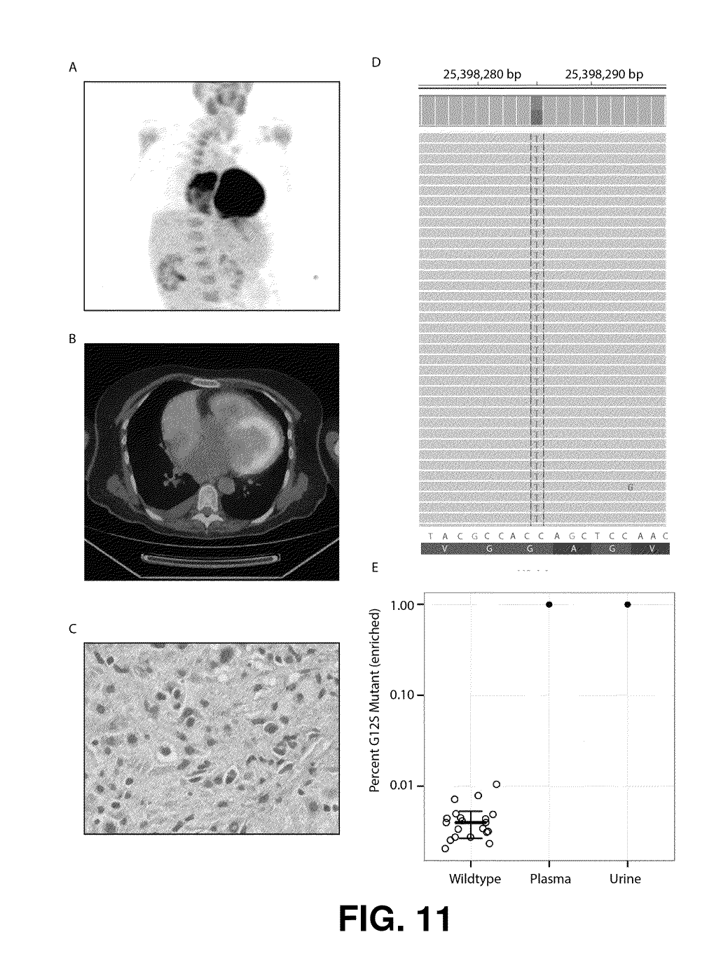

56.7% (17/30) of the patients enrolled in this study were identified as having a BRAF V600E mutation based on either tissue genotyping and/or cfDNA analysis. Identification of additional somatic mutations in BRAF V600E-wildtype patients is therefore of great importance for identifying targeted therapeutic strategies for those patients without BRAF mutations as well as for identifying markers to track disease in cfDNA. One BRAF wildtype patient here was found to have a KRAS G12S mutation in tissue material taken from a cardiac ECD lesion (FIG. 11A-D). This mutation was also found to be present by cfDNA analysis in both plasma and urine (FIG. 11E and Table 5). Although NRAS mutations have been reported in ECD18, KRAS mutations have never previously been reported in these disorders.

TABLE 5
Plasma and urine cfDNA mutation detection z-scores for KRAS
(G12A, G12C, G12D, G12R, G12S, G12V, G13D) with tissue biopsy
KRAS G12G from patient with Erdheim-Chester Disease (ECD).

| | G12A | G12C | G12D | G12R | G12S | G12V | G13D |
|---|---|---|---|---|---|---|---|
| Tissue | Neg. | Neg. | Neg. | Neg. | Pos. | Neg. | Neg. |
| Biopsy | | | | | | | |
| Plasma | −0.54 | −2.44 | −5.71 | 0.18 | 69.77 | −1.25 | −0.45 |
| cfDNA | | | | | | | |
| Urine | −0.60 | −2.65 | −8.19 | −0.34 | 70.05 | −1.71 | −1.36 |
| cfDNA | | | | | | | |

### Discussion

This study confirms the demonstration, in Examples 1-6, of the utility of circulating cfDNA for reliably detecting actionable alterations and monitoring response to therapy in histiocytic disorder patients. A high correlation of tissue mutational genotype was identified with urine and plasma cfDNA mutational status, establishing the utility of cfDNA mutational assessment of BRAF V600E mutations in LCH and ECD patients. Moreover, quantitative BRAF V600E cfDNA allele burden changed dynamically with therapy and mirrored radiographic evaluation of disease. These findings have potentially important implications for the initial diagnostic workup and serial monitoring of these rare disorders.

We found that 30% of patients ( 9/30) had an indeterminate BRAF mutation result from tumor tissue despite concerted genotyping efforts. This high proportion of patients with unknown tissue biopsy genotype underscores the substantial difficulty in identifying tumor genotype information in histiocytic disorder patients. The high proportion of BRAF genotyping test failures here likely relates to the frequent use of bone as a site of biopsy in these disorders. Eight of the 9 (88.9%) patients with an initial unknown BRAF tissue genotyping status had biopsies from bone. The molecular assessment of bony lesions is challenging as morphologic assessment requires decalcification procedures that often render the tissue unsuitable for molecular testing. Furthermore, aspirates of these lesions often yield suboptimal material for testing, with findings of non-specific inflammation and/or fibrosis and low histiocyte content. Of the 9 patients with indeterminate BRAF genotype from tissue biopsy, cfDNA testing identified BRAF mutations in 2 patients. These results have immediate therapeutic implications.

In addition to the use of cfDNA for establishing initial presence or absence of BRAF V600E mutations, serial measurements of BRAF V600E mutant allele burden on a variety of therapies revealed the utility of cfDNA analysis for dynamically monitoring response to both immunomodulatory and BRAF inhibitor therapy in these disorders. Assessment of treatment response has been an obstacle in the treatment of adult histiocytic disorder patients as radiographic assessments of response do not accurately characterize the wide spectrum of anatomic sites and lesion types characteristic of these disorders. Moreover, no formal criteria for assessment of treatment response exist for adult LCH and ECD patients. Thus, these data support incorporation of urinary and/or plasma cfDNA allele burden as a potential surrogate marker for clinical benefit in future clinical trials and standard of care of histiocytic disorder patients. It is important to note that the rate of decline in the BRAF mutant allele burden in urinary and plasma cfDNA is variable between patients, underlining the need for multiple serial assessments of allele burden following initiation of therapy. Also, given that cfDNA BRAF V600E mutation detection mirrored response to multiple therapeutic modalities here, it is likely that cfDNA detection of BRAF mutations may serve as a good marker of disease burden in histiocytosis patients rather than solely serving as a therapeutic target.

The use of urine as the source of cfDNA as reported here particularly facilitated routine serial monitoring of BRAF V600E allele burden. While somatic mutation detection has been performed in cfDNA of cancer patients previously, nearly all prior studies utilizing urinary cfDNA in cancer were restricted to patients with genitourinary malignancies (Casadiao et al., 2014a, b; Zhang et al., 2012). However, urinary cfDNA detection of BRAF V600E mutations mirrored closely the results from plasma cfDNA analysis here. Moreover, as shown in FIG. 10, urinary samples for cfDNA could be obtained on a weekly basis allowing for disease monitoring on an outpatient basis without the need for phlebotomy or other medical procedures. Previous studies indicate that DNA in urine can be stabilized for at least nine days (Zhang et al., 2012), whereas plasma requires processing within six hours for accurate assessment of cfDNA (Chan et al., 2005).

Combined use of tissue and cfDNA genotyping analyses also allowed the identification of a KRAS mutation in BRAF wildtype ECD patients (a mutation not previously described in ECD). Future interrogation of RAS mutations in tumor biopsies and cfDNA from BRAF wildtype histiocytic disorder patients may provide an additional somatic mutational biomarker and therapy options in this patient population.

Overall, these data confirm the findings in Examples 1-6 that monitoring of BRAF V600E mutations in cfDNA of histiocytic disorder patients provides a reliable and convenient noninvasive method to detect BRAFV600E mutations and assess treatment response in these unique disorders.

### REFERENCES

Aouba et al., 2010, Blood 116:4070-6.
Arceci R J, 2014, Am Soc Clin Oncol Educ Book. 2014:e441-5.
Arcila et al., 2011, J Mol Diagn 13:64-73.
Arnaud et al., 2012, Rheumatology (Oxford) 51:948-50.
Arnaud et al., 2011, Blood 117:2778-2782.
Badalian-Very et al., 2010, Blood 116:1919-23.
Berres et al., 2014, J Exp Med 211:669-83.
Breiman et al., 1984, Classification and Regression Trees, Chapman & Hall, 1984.
Brown et al., 2014, Blood 124:1655-8.
Cangi et al., 2014, Ann Rheum Dis. 2013-204924. (Epub)
Casadio et al. 2013a, Biomed Res Int 2013:270457.
Casadio et al., 2013b, Urol Oncol 31:1744-50.
Chakraborty, 2014, Blood-2014-05-577825 (Epub)
Chan et al., 2005, Clin Chem 51:781-4.
Crowley et al., 2013, Nat Rev Clin Oncol. 10:472-484.
Dawson et al., 2013, N Engl J Med. 368:1199-1209.
De Mattos-Arruda et al., 2013, Nat Rev Clin Oncol. 10:377-389.
Diamond et al., 2013, Blood 122:1089-91.
Diaz et al., 2012, Nature 486:537-540.
Diehl et al., 2008, Nat Med. 14:985-990.
Emile et al., 2013, J Clin Oncol. 31:398.
Emile J F, 2014, Blood-2014-04-570937 (Epub)
Forshew et al., 2012, Science Translational Medicine, 4:136ra168.
Frampton et al., 2013, Nat Biotechnol 31:1023-31.
Girschikofsky et al., 2013, Orphanet J Rare Dis 8:72.
Go et al., 2014, Histopathology 65:261-72.
Haroche et al., 2012, Blood 120:2700-3.
Haroche et al., 2013, Blood 121:1495-1500.
Hatzivassiliou et al., 2010, Nature 464:431-435.
Janku et al., 2010, J Clin Oncol. 28:e633-636.
Janku et al., 2013, Oncologist 18:2-4.
Janku et al., 2014, Oncotarget 5: 3607-3610.
Keshet Y and Seger R., 2010, Methods Mol Biol. 661:3-38.
Malo et al., 2006, Nat Biotechnol 24:167-75.
Misale et al., 2012, Nature 486:532-536.
Murtaza et al., 2013, Nature. 497:108-112.
Niault T and Baccarini M., 2010, Carcinogenesis. 31:1165-74.
Sahm et al., 2012, Blood 120:e28-34.
Satoh et al., 2012, PLoS One 7:e33891.
Su et al., 2008, Annals of the New York Academy of Sciences 1137:197-206.
Wan et al., 2004, Cell 116:855-67.
Wellbrock C and Hurlstone A, 2010, Pharmacol. 80:561-7.
Zhang et al., 2012, Genet Mol Res 11:3007-12.
U.S. Pat. No. 8,623,603.
US Patent Application Publication US2010/0068711.
U.S. Provisional Patent Application No. 62/039,905.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Moreover, their citation is not an indication of a search for relevant disclosures. Applicants reserve the right to challenge the accuracy and pertinence of the cited references.

What is claimed is:

1. A method of detecting a mutation in a histiocytosis patient, the method comprising
(a) obtaining a sample of a bodily fluid from the patient; and
(b) testing the sample for the presence of a mutation in a gene in the RAS-RAF-MEK-ERK or the RAS-PI3K-AKT pathway in cell free DNA (cfDNA) in the bodily fluid.

2. The method of claim 1, wherein the bodily fluid is serum or plasma.

3. The method of claim 1, wherein the bodily fluid is urine.

4. The method of claim 1, wherein the mutation is in the BRAF, KRAS, PIK3A, NRAS, MAPK1, ARAF or ERBB3 genes.

5. The method of claim 1, wherein the mutation is BRAF V600E.

6. The method of claim 1, wherein the mutation is a KRAS mutation.

7. The method of claim 6, wherein the KRAS mutation is G12A, G12C, G12D, G12R, G12S, G12V or G13D.

8. The method of claim 1, wherein the histiocytosis is Langerhans Cell Histiocytosis (LCH).

9. The method of claim 1, wherein the histiocytosis is non-Langerhans Cell Histiocytosis (nLCH).

10. The method of claim 9, wherein the nLCH is Erdheim-Chester Disease (ECD).

11. The method of claim 1, wherein the testing comprises sequencing.

12. The method of claim 1, wherein the testing comprises polymerase chain reaction (PCR).

13. The method of claim 12, wherein the PCR is droplet digital PCR.

14. The method of claim 12, wherein the PCR amplifies a sequence of less than about 100 nucleotides.

15. The method of claim 12, wherein the PCR is performed using a blocking oligonucleotide that suppresses amplification of a wildtype version of the gene.

16. The method of claim 1, wherein, if the mutation is present, the patient is treated with a medicament that targets the product of the gene having the mutation.

17. A method of monitoring disease course of a histiocytosis in a patient having a mutation in a gene in the RAS-RAF-MEK-ERK or the RAS-PI3K-AKT pathway, the method comprising
(a) obtaining a first DNA-containing sample from the patient;
(b) quantifying the mutation and its corresponding wildtype sequence in the first sample at a first time point; and
(c) repeating (a) and (b) at a second time point with a second sample,
wherein an increase in the quantity of the mutation relative to its corresponding wildtype between the first and second time point indicates that the histiocytosis is progressing, and a decrease in the quantity of the mutation relative to its corresponding wildtype indicates that the histiocytosis is remitting.

18. A method of selecting and/or applying treatment or therapy for a histiocytosis patient, the method comprising
detecting a mutation in the patient by the method of claim 1; and
selecting and/or applying a treatment or therapy based on the detecting.

19. A method of selecting and/or applying treatment or therapy for a histiocytosis patient, the method comprising
monitoring progression of the histiocytosis in the patient by the method of claim 17; and
selecting and/or applying a treatment or therapy based on the monitoring.

20. A method of treating a patient having a histiocytosis, the method comprising
(a) testing for the quantity of a mutation in a gene in the RAS-RAF-MEK-ERK or the RAS-PI3K-AKT pathway and a corresponding wildtype sequence in DNA-containing samples taken from the patient at a plurality of time points;
(b) determining whether the quantity of the mutation relative to its corresponding wildtype sequence increased from an earlier time point to a later time point; and
(c) selecting and/or applying a treatment or therapy based on the determining.


SEQUENCE LISTING (2 sequences)

SEQ ID NO: 1 - DNA, 2946 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttcttttaaaaa

SEQ ID NO: 2 - protein, 766 aa, Homo sapiens [sequence omitted]